(19) United States
(12) Patent Application Publication
(10) Pub. No.: US 2026/0035333 A1
(43) Pub. Date: Feb. 5, 2026

(54) PROCESS FOR PURIFYING BIS(2-HYDROXYETHYL)TEPHTHALATE OBTAINED FROM DEPOLYMERIZATION OF WASTE CONTAINING POLYETHYLENE TEREPHTHALATE

(71) Applicant: CHEMPET SRL, Cerano (NO) (IT)

(72) Inventors: Guido FRAGIACOMO, Novara (NO) (IT); Beatrice MASSACCESI, Foligno (PG) (IT); Marco CASAROTTI, Galliate (NO) (IT); Roberto PROSERPIO, Mariano Comense (CO) (IT)

(73) Assignee: CHEMPET SRL, Cerano (NO) (IT)

(21) Appl. No.: 19/101,296

(22) PCT Filed: Jul. 31, 2023
(86) PCT No.: PCT/IB2023/057756

§ 371 Date: Feb. 5, 2025

(30) Foreign Application Priority Data
Aug. 5, 2022 (IT) 102022000016851

(51) Int. Cl. C07C 67/52

(52) U.S. Cl. CPC C07C 67/52

(57) ABSTRACT

Process for purifying his (2-hydroxyethyl) terephthalate (BHET) from a crude solution, obtained from the depolymerization of a polyethylene terephthalate (PET) waste containing at least one barrier-polymer by glycolysis with ethylene glycol (EG), to which water is added, which acts as an anti-solvent for the barrier-polymers, in predetermined quantities and the temperature is controlled in such a way as to ensure a significant reduction in the solubility of the barrier polymers without causing precipitation of BHET and oligomers thereof. In particular, water is added to the crude BHET solution in at least two distinct phases with decreasing weight ratios between EG and H₂O and also decreasing temperature. In this way, the removal of the barrier-polymers is more effective and does not cause precipitation of BHET and its oligomers. Furthermore, in this way, it is possible to keep the volume of the liquid to be treated within relatively reduced values, so as to avoid plant drawbacks associated with the treatment of a large volume of liquid by decantation and/or filtration, which would make the process inconvenient from an industrial point of view.

The present invention relates to a process for purifying bis(2-hydroxyethyl) terephthalate (BHET) obtained from the depolymerization of waste containing polyethylene terephthalate (PET).

Polyethylene terephthalate (PET) is a widely used semi-crystalline thermoplastic polyester having high strength and transparency, which has various applications due to its physical and chemical properties, especially in packaging and fiber production. PET does not involve any safety risk, but increase in consumption and accumulation in waste streams and its non-biodegradability generate environmental and economic concerns. Therefore, there is growing interest in PET recycling technologies.

PET is considered a polymeric material that can be easily recycled, and its recycling is the most widespread among polymeric materials. The technologies can be grouped into two macro-categories: mechanical and chemical recycling.

Mechanical recycling mainly consists of obtaining flakes, by sorting waste, removing contaminants, crushing and grinding, which are fed directly to extrusion to produce new articles. The main problems of this technology are due to the heterogeneity of solid waste and the low quality of the final product, since the properties of PET are degraded every time it is recycled.

Chemical recycling involves the decomposition of polyester using a reagent capable of depolymerizing the PET chains to obtain the starting monomers. The chemical depolymerization of PET is usually achieved by solvolysis, in particular by hydrolysis or methanolysis or glycolysis.

Hydrolysis depolymerizes PET into terephthalic acid (TPA) and ethylene glycol (EG) (also called monoethylene glycol—MEG) by reaction with water. Methanolysis degrades PET to dimethyl terephthalate (DMT) and EG by reaction with methanol. Glycolysis causes depolymerization by reaction with EG, to produce bis(2-hydroxyethyl) terephthalate (BHET), an intermediate product formed in a first step of PET production from the starting monomers.

To date, mechanical recycling is still the most widely used technology for the treatment of PET waste and there are only few industrial applications of chemical recycling. However, there is a growing interest in chemical recycling technologies as they comply with sustainable development principles, providing virgin PET as raw material, which is of course of a much higher quality than mechanically recycled PET.

To better understand the basics of chemical recycling, the following are the manufacturing fundamentals of PET and glycolysis.

Industrially, PET is obtained from two separate reaction steps. In the first step, the starting products react to produce BHETs and oligomers, and then chain elongation takes place in a separate reaction step.

Two different raw materials can be used to produce PET: (a) via acid, TPA and EG react to produce BHET and water; or (b) via ester, dimethyl terephthalate (DMT) and EG react to produce BHET and methanol. Subsequently, the BHET, produced through an esterification or transesterification step, reacts to produce PET through a polycondensation reaction; in this step, EG develops which can be separated from the reaction mixture:

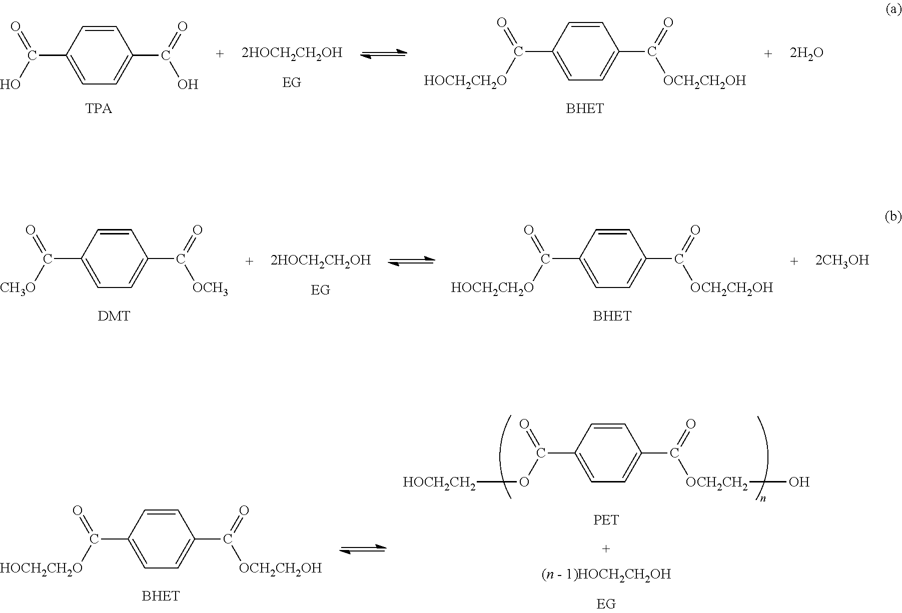

Polycondensation is an equilibrium reaction and EG is produced as a byproduct with PET. It is therefore necessary to shift the equilibrium towards PET by evaporating the developed EG.

The reverse reaction can be used to produce BHET from PET: the reaction between PET and EG results in chain scission by attacking the ester bond. Since PET is formed through a reversible polycondensation reaction, the polymer can be transformed into its monomer or oligomer by shifting the reaction in the opposite direction by adding EG.

During this reverse reaction, called glycolysis, the polymer chains are transformed by means of a solvolytic chain scission leading to a theoretically complete depolymerization up to the monomer (BHET) or a partial depolymerization leading to the monomer together with the oligomers:

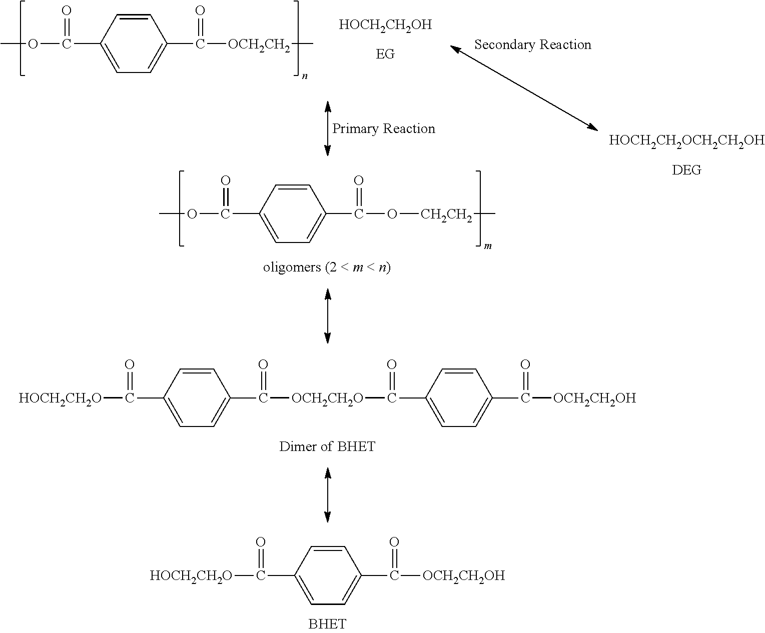

The reaction is slightly endothermic and reversible; therefore, it can be pushed towards the production of monomer/oligomers by working with an excess of EG compared to the starting PET.

The whole glycolysis reaction can be divided into two steps: (i) EG splits the ester bonds in the PET chain and the oligomers are formed and solubilized in the EG itself; (ii) the oligomers are in equilibrium with BHET, therefore the reaction moves towards the formation of BHET by adding an excess of EG.

Without using a catalyst, the glycolysis reaction is slow, and complete depolymerization of PET to BHET is difficult to achieve in an acceptable time for an industrial process. Therefore, a transesterification catalyst is usually used, which ensures shorter reaction times at lower temperatures than the non-catalyzed reaction, and an increase in yield is achieved by removing or minimizing any side reactions that can affect the whole process. Possible catalysts are described for example in U.S. Pat. No. 6,630,601 and WO 2017/111602. The catalyzed glycolysis reaction is usually performed at a temperature of 190° C. to 250° C.

In order to be used for the production of virgin PET, the BHET obtained from the glycolysis of PET waste must be carefully purified, as it contains various contaminants that can interfere with the polymerization reaction and/or deteriorate transparency and color of the produced PET.

For example, in patent application WO 2021/124149, in the name of the same Applicant, it is described a process for removing dyes or other soluble organic contaminants from crude BHET, obtained from PET glycolysis, which comprises an oxidation step and a step of treating the oxidized solution with an adsorbing agent, which adsorbs the oxidation products of the contaminants and is then removed to obtain the purified BHET solution.

BHET may also contain contaminants that are insoluble in the medium in which glycolysis is conducted, such as aluminum chips, polyolefins, fillers (such as titanium dioxide, carbon black, silicates and other pigments), adhesives, and the like. Insoluble contaminants can be separated by filtration.

Other contaminants are barrier-polymers that are used in the production of multilayer materials. In fact, to preserve products sensitive to atmospheric oxygen (in particular food, beverages, pharmaceutical or cosmetic products), multilayer materials based on PET are used coupled with barrier-polymer layers, which hinder oxygen penetration. In the case of carbonated drinks, the barrier-polymer layer hinders escape of carbon dioxide, while in the case of food it preserves aroma and therefore the organoleptic properties of the food itself.

The most widely used barrier-polymers are polyamides (PA), especially nylon-6 (PA-6), nylon-6,6 (PA-6,6), polyvinyl alcohol (PVA), polyvinyl acetate (PVAc), ethylene copolymers/vinyl alcohol (EVOH), and the like.

Generally, these multilayer materials comprise a polymer-barrier layer, a heat-sealable sealing layer (for example in polyethylene) and an internal layer of PET, which comes into direct contact with the product to be preserved.

The Applicant has faced the problem of purifying bis(2-hydroxyethyl)terephthalate (BHET) obtained by depolymerization of PET waste via glycolysis with EG from the barrier-polymers with which PET is coupled as described above. The product obtained from depolymerization via glycolysis is a crude solution or suspension in which BHET and its oligomers (especially dimers and trimers) are dissolved in EG together with different types of contaminants which are solid or dissolved in EG as well, namely:
(a) contaminants soluble in EG or which react with EG so as to solubilize;
(b) contaminants which remain solid because they are insoluble or only partially soluble in EG at the glycolysis temperature;
(c) contaminants that do not react with EG but are soluble in EG at the glycolysis temperature.

To category (a) belong for example organic dyes, antistatic agents, stabilizers, ultraviolet ray absorbers, which can be removed by the process described in the above-mentioned application WO 2021/124149.

Solid contaminants at glycolysis temperature (category (b)), such as fillers (e.g. titanium oxide, carbon black, silica), adhesives, pigments, aluminum films or fragments, polyolefins, are separated from the crude BHET solution by filtration of the material that accumulates on the bottom of the glycolysis reactor (down-flow), or by skimming for contaminants having a density lower than the crude glycolized product which float on the surface (this is the case of polyolefins). In this regard, see for example U.S. Pat. No. 6,410,607.

The barrier-polymers described above belong to category (c). As suggested in patent application WO 2021/124149 cited above, the polyamides can be separated from the crude BHET solution by adding water, which favors polyamide precipitation, while the BHET and its oligomers remain in solution. The precipitated polyamides can then be separated by filtration.

The Applicant has found that, in order to obtain an effective separation of polyamides and other barrier-polymers from the BHET crude solution, it is necessary to carry out an accurate water dosage, which acts as an anti-solvent for the barrier-polymers, and a temperature control such as to ensure a significant reduction in the solubility of the barrier-polymers without causing precipitation of BHET and its oligomers, which remain in solution.

In particular, the Applicant has found that, in order to obtain a substantially complete removal of the barrier-polymers, water must be added to the crude BHET solution in at least two distinct steps with decreasing weight ratios between EG and H₂O and decreasing temperature as better defined below. In this way the barrier-polymer removal is more effective and does not cause precipitation of BHET and its oligomers. In other words, the at least partial precipitation of the barrier-polymers is carried out in a separate phase with respect to the separation of the oligomers, which can be recycled to the glycolysis reaction. Furthermore, the volume of the liquid to be treated can be kept within relatively low values, so as to avoid plant drawbacks associated with the treatment of a large volume of liquid by decantation and/or filtration, which would make the process inconvenient from an industrial point of view.

Therefore, according to a first aspect, the present invention relates to a process for purifying bis(2-hydroxyethyl)terephthalate (BHET), which comprises:
providing a crude BHET solution obtained from depolymerizing a polyethylene terephthalate (PET) waste by glycolysis with ethylene glycol (EG), said crude BHET solution being at a temperature from 160° C. to 220° C., preferably from 190° C. to 210° C., and containing at least one dissolved barrier-polymer;
adding a first portion of water to said crude BHET solution in an amount such as to obtain a weight ratio EG:H₂O from 2.0 to 4.0, preferably from 2.5 to 3.5, and cooling the solution thus obtained to a temperature from 60° C. to 90° C., preferably from 65° C. to 80° C., so as to obtain a precipitate of said at least one barrier-polymer;
separating said precipitate of at least one barrier-polymer from the BHET solution;
adding a second portion of water to the BHET solution obtained from the separation step in an amount such as to obtain a weight ratio EG:H₂O from 0.5 to 2.5, preferably from 1.0 to 2.0, and cooling the solution thus obtained to a temperature ranging from 5° C. to 25° C., preferably from 10° C. to 20° C., so as to obtain the crystallization of the BHET;
separating the crystallized BHET from the water/EG solvent.

Preferably, said PET waste is a multilayer product comprising at least one PET film coupled with at least one polymer barrier film.

As for the crude BHET solution, it is obtained from the depolymerization of PET waste by a glycolysis reaction with EG.

The glycolysis reaction is usually performed in the presence of a heterogeneous transesterification catalyst. The catalyst can be selected, for example, from: carbonates, fatty acid salts or borates of Na, Mg, Zn, Cd, Mn, Co, Ca or Ba (e.g. zinc borate, zinc acetate, sodium carbonate).

Preferably, the glycolysis reaction is carried out at a temperature of 170° C. to 270° C., more preferably 195° C. to 210° C.

In the glycolysis reaction, EG is generally used in an amount of 1.0 to 10.0 parts by weight, preferably 1.5 to 6.0 parts by weight, based on the parts by weight of the PET waste.

The duration of the glycolysis reaction can vary within wide ranges, depending on the reaction conditions, such as temperature, agitation, reactor type and the like. Usually the reaction time is from 1 to 8 hours, preferably from 1.5 to 3 hours. The reaction can be performed in batches or continuously. The reaction pressure is usually atmospheric pressure, however reduced or increased pressure may be used.

Further details relating to the glycolysis reaction of a PET waste to recover BHET are reported, for example, in U.S. Pat. Nos. 3,222,299, 4,609,680 and EP 0 723 951 A1.

The product obtained from the glycolysis reaction is a solution of crude BHET, where the BHET is dissolved in EG together with various contaminants which arise from the specific composition of the PET waste. Usually, the crude BHET solution also contains BHET oligomers, preferably dimers and/or trimers.

In the present description and the appended claims, the total amount of BHET in a BHET solution is calculated on the basis of the BHET (monomer) and all its oligomers.

At least one barrier-polymer is present in the raw BHET solution, i.e. a polymer, generally in the form of a film, which is capable of hindering the passage of gases or vapors, in particular oxygen, carbon dioxide and of flavors present in foods.

In particular, the barrier-polymer is selected from:
(a) polyamides (PA), in particular nylon-6 (PA-6), nylon-6,6 (PA-6,6), and copolymers thereof (for example the polyamides marketed by BASF AG under the brand name Ultramid®);
(b) polyvinyl alcohol (PVA), polyvinyl acetate (PVAc), ethylene/vinyl alcohol copolymers (EVOH), ethylene/vinyl acetate copolymers (EVA), ethylene/vinyl acetate/vinyl alcohol terpolymers;
or mixtures thereof.

In addition to the barrier-polymers mentioned above, the contaminants present in the raw BHET solution can be for example:
dyes, usually organic dyes;
inks;
adhesives and glues;
polyolefins, for example polyethylene or polypropylene used to produce caps;
PET-G;
biodegradable polymers, for example PLA;
UV absorbers;
fillers, for example titanium dioxide, carbon black, silica, silicates and other pigments;
metal sheets and their fragments, for example aluminum sheets.

Following addition of the first portion of water and cooling of the crude BHET solution, the barrier-polymers present in the solution itself at least partially precipitate, and can therefore be separated from the solution, together with the other insoluble contaminants, for example by filtration. Filtration can be carried out for example with a filter press.

In the event that the BHET solution thus obtained contains non-negligible quantities of BHET oligomers which are to be separated from the BHET monomer, after separating the barrier-polymers and other insoluble contaminants that may be present, the BHET solution can be further cooled to a temperature from 30° C. to 60° C., preferably from 35° C. to 55° C., so as to precipitate the oligomers, while maintaining the BHET monomer in solution. In this phase, in case the barrier-polymers have not been completely separated in the previous phase, the above cooling can cause the co-precipitation, together with the oligomers, of the barrier-polymers remaining in solution. The precipitated oligomers, together with the barrier-polymers, can then be separated from the solution, for example by filtration or centrifugation, for example by means of a horizontal axis centrifuge (decanter). The oligomers thus obtained can optionally be recycled to the initial glycolysis reaction, together with the co-precipitated barrier-polymers.

The BHET solution, from which the barrier-polymers, the other insoluble contaminants and possibly the oligomers have been removed, is then added with a second portion of water in such an amount as to obtain a weight ratio EG:H₂O from 0.5 to 2.5, preferably from 1.0 to 2.0, and further cooled to a temperature of from 5° C. to 25° C., preferably from 10° C. to 20° C., so as to obtain the crystallization of the BHET.

The addition of the second portion of water has the main purpose of favoring the crystallization of the BHET and its subsequent separation. Furthermore, the quantity of BHET which remains dissolved in the water/EG solvent is minimized, which can in any case be subsequently recovered as a distillation bottom of the water/EG mixture.

The crystallized BHET can then be separated from the water/EG solvent, for example by filtration or other conventional methods, in particular by vacuum filtration.

The EG present in the water/EG mixture can advantageously be recovered, for example by fractional distillation, while the BHET can be subjected to further purification steps.

Preferably, the process according to the present invention further comprises:
dissolving crystallized BHET in water;
treating the thus obtained solution with at least a first adsorbing agent;
separating the at least one first adsorbing agent from the so treated solution so as to obtain a purified first solution of BHET.

The adsorbing agent has the function of adsorbing any residual barrier-polymers or other insoluble contaminants that remain in the crystallized BHET. In this way the quantity of residual contaminants (in particular of organic dyes) is reduced, so as to reduce the quantity of oxidizing agent necessary for the subsequent purification of the BHET and therefore of the reduction products of the same (for example chlorides in case a hypochlorite is used as an oxidizing agent).

The adsorbing agent is a solid material, usually in the form of a powder, which is capable of adsorbing organic molecules dispersed or solubilized in a liquid medium on its surface, in order to sequester and eliminate these molecules by separating the adsorbing agent from the liquid medium.

Preferably, the adsorbing agent is an activated carbon or a silica. Preferably, the adsorbing agent has a particle size of 50 to 500 mesh, more preferably 100 to 350 mesh, measured according to ASTM D2862/16 standard.

Preferably, the adsorbing agent has a surface area (BET) of 250 to 5000 m²/g, more preferably of 500 to 3000 m²/g, measured according to ASTM D6556/19 standard.

The quantity of adsorbing agent to be added depends mainly on its nature and on the concentration of the contaminants to be separated. For example, in the case of activated carbon, it is preferably added in an amount of from 0.05% to 3% by weight, more preferably from 0.1% to 1% by weight, with respect to the weight of the BHET present in the solution to be purified. The purification step is preferably carried out at a temperature of from 40° C. to 100° C., more preferably from 70° C. to 90° C.

After the treatment with the first adsorbing agent, the separation of said first adsorbing agent from the solution is performed so as to obtain a purified first solution of BHET. This separation can be performed according to known techniques, preferably by filtration.

In order to eliminate further contaminants from the first purified solution of BHET thus obtained, in particular organic dyes and other low molecular weight organic contaminants, the process according to the present invention preferably further comprises:
treating the solution obtained after the separation of the adsorbing agent with at least one oxidizing agent at a temperature from 30° C. to 100° C., preferably from 50° C. to 90° C., to obtain an oxidized solution;
treating the oxidized solution with at least a second adsorbing agent to obtain a purified oxidized solution;
separating said at least one second adsorbing agent from the purified oxidized solution to obtain a second purified BHET solution.

The second adsorbing agent, equal to or different from the first, has features selected from those indicated above for the first adsorbing agent.

The oxidizing agent is preferably selected from inorganic oxidizing agents, such as: alkali metal or alkaline earth metal chlorites or hypochlorites; alkali metal or alkaline earth metal persalts, preferably persulfates; potassium permanganate; hydrogen peroxide; ozone; gaseous chlorine; or mixtures thereof. Preferably, the alkali metal is sodium or potassium, and the alkaline earth metal is magnesium, calcium or barium.

The following are particularly preferred: sodium or potassium hypochlorite or chlorite; hydrogen peroxide; or mixtures thereof.

To improve effectiveness of hydrogen peroxide, it can be used in combination with UV radiation. For this purpose, the treated solution is irradiated with a UV lamp, usually with a radiation wavelength of 150 nm to 400 nm.

The oxidizing agent is preferably used in an amount of 0.005% to 5% by weight, preferably 0.01% to 3% by weight, based on the BHET weight in the solution.

Further details on the purification of the BHET solution by treatment with an oxidizing agent can be found in the patent application WO 2021/124149, already mentioned above.

The BHET, and possibly present oligomers thereof, can be recovered from the first or second solution of BHET purified preferably by precipitation. To this end, the solution can be cooled to a temperature of 5° C. to 30° C., more preferably 10° C. to 20° C. BHET and its oligomers can precipitate in an amorphous state or in an at least partially crystalline form, mainly depending on the conditions applied for the precipitation.

The thus obtained purified BHET can then be subjected to washing with water and final drying.

The following examples are provided for purely illustrative purposes of the present invention and should not be considered as a limitation of the scope defined by the appended claims.

### Examples 1a-1b-1c

A series of tests were carried out on PET waste obtained from bottles (r-PET) with the addition of various polyamides used as barrier-polymers for food packaging. The r-PET was in the form of clear and clean flakes, substantially free of insoluble contaminants.

In each sample, the amount of added polyamide was equal to 5% by weight with respect to the weight of r-PET. The amount of polyamide in the suspension subjected to glycolysis was equal to 1% by weight.

The following polyamides were used:
PA-6 produced by Aquafil S.p.A. (Example 1a);
PA-6,6 produced by Aquafil S.p.A. (Example 1b);
PA-6 Ultramid® B grade produced by BASF AG (Example 1c).

PET waste with added PA was dissolved in EG at 200° C., under reduced pressure (p=0.4 MPa). The glycolysis reaction was performed in the presence of zinc acetate (ZnAc) as a heterogeneous catalyst (0.1% by weight with respect to PET weight). The glycolysis temperature was 207° C. for Example 1a and 204° C. for Examples 1b and 1c. For all examples, glycolysis was carried out in two phases with different PET/EG ratio: a first phase of 40 min with a PET/EG ratio equal to 1, followed by a second phase of 30 min with a PET/EG ratio equal to 4.

Table 1 shows the quantities of products used for the three glycolysis reactions.

TABLE 1

| | Example | | | | | |
|---|---|---|---|---|---|---|
| | 1a | | 1b | | 1c | |
| | weight | % by | weight | % by | weight | % by |
| | (g) | weight | (g) | weight | (g) | weight |
| r-PET | 200 | 19.8 | 285 | 19 | 285 | 19 |
| EG | 800 | 79.2 | 1200 | 80 | 1200 | 80 |
| ZnAc | 0.2 | 0 | 0.3 | 0 | 0.3 | 0 |
| PA-6 | 10 | 1 | — | — | — | — |
| Aquafil | | | | | | |
| PA-66 | — | — | 15 | 1 | — | — |
| Aquafil | | | | | | |
| PA-6 | — | — | — | — | 15 | 1 |
| Ultramid | | | | | | |
| Total | 1010.2 | 100 | 1500.3 | 100 | 1500.3 | 100 |

The crude BHET solutions obtained from the above glycolysis processes were subjected to the purification process according to the present invention. The different steps of the process with the specific conditions used are shown below:
(a) the crude solution obtained from glycolysis was cooled to 110° C. and then added with water (ratio EG:H₂O=3);
(b) the solution obtained from step (a) was cooled to 75° C. in order to obtain a partial precipitation of the PA;
(c) the solution obtained from step (b) was filtered through a laboratory filter to separate the precipitated PA;
(d) the filtered solution obtained from step (c) was cooled to 50° C. to obtain precipitation of the BHET oligomers, which were separated by means of a horizontal axis centrifuge (decanter); together with the BHET oligomers, a residual portion of PA remained in solution is co-precipitated, which was also separated together with the oligomers;
(e) water was added to the obtained solution after separation of the oligomers and of the PA according to step (d) in such an amount as to obtain a ratio EG:H₂O=1.5;
(f) the solution obtained from step (e) was cooled to a temperature of 15° C. in order to obtain the precipitation of BHET in crystalline form;
(g) the BHET precipitate was separated from the mother liquors (EG/H₂O mixture) by filtration with a laboratory filter;
(h) the BHET was dissolved in water and added with activated carbon (PAC) in an amount equal to 0.25% by weight with respect to the BHET weight (characteristics of the PAC: particle size: 325 mesh; BET surface area: approx. 2000 m²/g);
(i) the BHET solution containing the PAC in suspension was kept under stirring for 30 min at a temperature of 70°−75° C.; the PAC and the residual fraction of PA were then removed by filtration;
(j) the first purified BHET solution obtained from step (i) was then subjected to oxidation with sodium hypochlorite (17% by weight aqueous solution) in an amount equal to 0.25% by weight with respect to the weight of the BHET, at temperature of 70°−75° C. for a reaction time of 40 min;
(k) the oxidized solution obtained from step (j) was treated with the same PAC under the same conditions of steps (h) and (i) so as to obtain a second solution of purified BHET, from which the BHET was separated by crystallization by cooling to 15° C.;
(l) The recrystallized BHET was filtered, washed with water and dried.

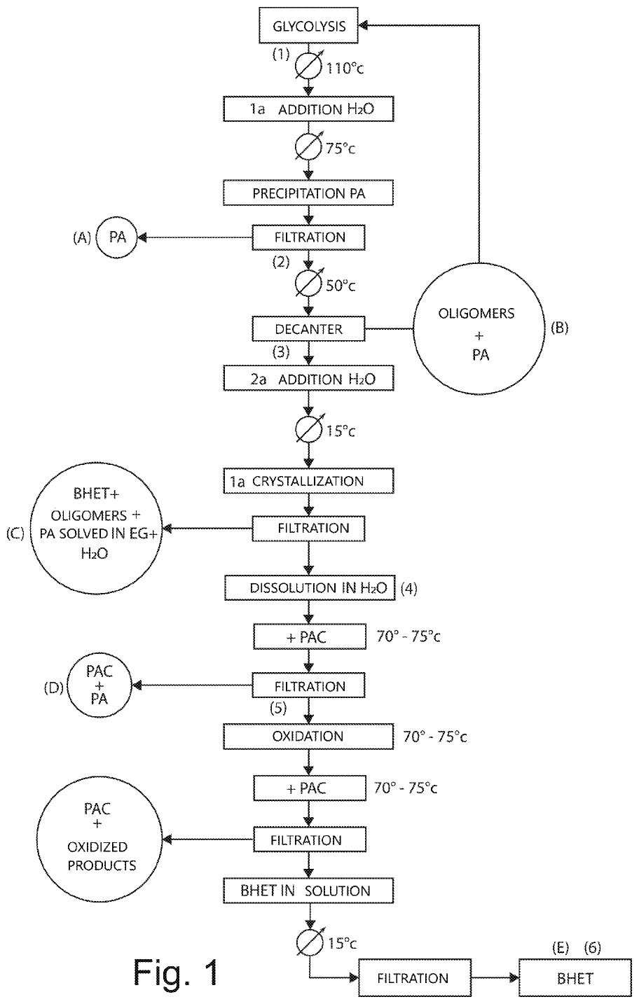

The scheme of the process is shown in FIG. 1.

### Analysis to Verify and Quantitatively Determine the Polyamide Removal

To verify the effectiveness of the process in separating the polyamides, two different analytical techniques were used: (i) by FT-IR spectrum to verify the presence of the polyamides by means of their characteristic absorption bands; (ii) by means of gravimetric measurements of the quantity of PA eliminated in the various steps of the process (mass balance).

These analyzes were carried out on the following products obtained in different steps of the process (shown in the process diagram of FIG. 1):
(A) filtered solid obtained from step (c);
(B) filtered solid obtained from step (d);
(C) mother liquors obtained from step (g);
(D) filtered solid obtained from step (i);
(E) final BHET obtained from step (1).

The following table shows the positions of the IR absorption bands (v, cm-1) characteristic of the used polyamides.

TABLE 2

| | PA-6 | PA-66 | PA-6 ULTRAMID |
|---|---|---|---|
| N—H amide stretching | 3291 | 3293 | 3296 |
| C═O amide stretching | 1634 | 1630 | 1633 |
| N—H amide bending | 1535 | 1534 | 1534 |

The presence of these bands made it possible to verify, within the sensitivity limits of the instrument, the presence or absence of polyamides in the various products (A)-(E) above.

For the quantitative evaluation of the polyamides separated in the various phases of the process as reported above, the procedure was as follows.

As regards the filtered solid (A), this essentially consisted of PA, since the r-PET used was substantially free of insoluble contaminants.

The filtered solid (B) essentially contained the oligomers and a portion of the residual PA not previously separated. The PA weight was determined as difference with respect to the initial weight, after separation of the oligomers by dissolving them with a water/methanol mixture.

The mother liquors (C) consisted of small quantities of BHET and its oligomers and of PA, dissolved in a EG/H₂O mixture. To determine the quantity of PA present in the mother liquors (C), precipitation of the solutes was carried out, which were separated and weighed by means of a thermoscale. A sample of the precipitate was weighed, BHET and oligomers were solubilized and the solution was analyzed by HPLC to determine its composition. The amount of PA was calculated as difference.

As regards the filtered solid (D), this was dried and weighed: since the amount of added PAC is known, the amount of PA was determined by difference.

In the final BHET (E) PA was no longer present, as demonstrated by the IR spectra of the product, which did not show the PA characteristic bands.

The results are summarized in the following Tables 3A-3B-3C, where the quantity of separated polyamide present in each sample is reported with respect to the total quantity added to the r-PET, expressed both as quantity by weight and as % by weight with respect to the total weight added to r-PET subjected to glycolysis. The presence or absence of the characteristic IR peaks are also reported (for the liquid C product the FT-IR spectrum was not determined). It should be noted that the position of the characteristic peaks measured on the pure polymers underwent a slight shift (<1%) due to the hydrogen bonds established between H₂O and EG and the polar groups of the polymers.

TABLE 3A

| | PA-6 | | |
|---|---|---|---|
| | | Separated | Separated PA (% by |
| Sample | IR peaks | PA (g) | weight of total PA) |
| A | yes (3255-1650-1550 cm⁻¹) | 5 | 50 |
| B | yes (1550 cm⁻¹) | 0.2 | 2 |
| C | — | 3 | 30 |
| D | yes (3255-1650-1550 cm⁻¹) | 1.8 | 18 |
| E | no | 0 | 0 |

TABLE 3B

| | PA-66 | | |
|---|---|---|---|
| | | Separated | Separated PA (% by |
| Sample | IR peaks | PA (g) | weight of total PA) |
| A | yes (3255-1650-1550 cm⁻¹) | 6.9 | 46 |
| B | yes (1550 cm⁻¹) | 0.5 | 3.3 |
| C | — | 5 | 33.3 |
| D | yes (3255-1650-1550 cm⁻¹) | 2.6 | 17.4 |
| E | no | 0 | 0 |

TABLE 3C

| | ULTRAMID PA-6 | | |
|---|---|---|---|
| | | Separated | Separated PA (% by |
| Sample | IR peaks | PA (g) | weight of total PA) |
| A | no | 0.15 | 1 |
| B | yes (3300-1630 cm⁻¹) | 1.8 | 12 |
| | yes (1550 cm⁻¹) | | |
| C | — | 5.85 | 39 |
| D | yes (3300-1630 cm⁻¹) | 7.2 | 48 |
| E | no | 0 | 0 |

As can be seen, polyamides were eliminated in a quantitative way. While the polyamides PA-6 and PA-6,6 were largely eliminated during step (c), i.e. after the first precipitation and subsequent filtration, only partial removal was achieved during step (c) in the case of Ultramid PA-6 polyamide, which was completed with subsequent steps, especially after the first treatment with PAC (step (i)).

### Analysis to Determine the Composition of BHET and Oligomers Thereof

The composition in terms of BHET and oligomers thereof at different steps of the process was also determined. The quantitative analysis was performed by HPLC on a sample taken from the process flow of the following products:
(1) crude solution obtained from glycolysis;
(2) filtered solution obtained from step (c);
(3) filtered solution obtained from step (d);
(4) BHET precipitate obtained from step (g) dissolved in a water:methanol mixture in a weight ratio of 65:35;
(5) first purified BHET solution obtained from step (h);
(6) crystallized, washed and dried BHET obtained from step (1) dissolved in the same water:methanol mixture as above.

The % by weight of BHET, dimer, trimer and other oligomers obtained for the different products (1)-(6) with the amides PA-6, PA-66 and Ultramid PA-6 are reported in Tables 4A, 4B and 4C.

TABLE 4A

PA-6

| Sample | BHET | Dimer | Trimer | Other oligomers |
|---|---|---|---|---|
| 1 | 89.26 | 9.32 | 0.76 | 0.66 |
| 2 | 92.16 | 7.06 | 0.39 | 0.39 |
| 3 | 97.64 | 1.81 | 0 | 0.55 |
| 4 | 96.68 | 2.59 | 0.06 | 0.67 |
| 5 | 96.68 | 2.59 | 0.06 | 0.67 |
| 6 | 97.67 | 1.69 | 0.02 | 0.62 |

TABLE 4B

PA-66

| Sample | BHET | Dimer | trimer | Other oligomers |
|---|---|---|---|---|
| 1 | 90.17 | 8.23 | 0.57 | 1.03 |
| 2 | 90.76 | 7.78 | 0.49 | 0.97 |
| 3 | 96.56 | 2.34 | 0.06 | 1.05 |
| 4 | 96.38 | 2.98 | 0.07 | 1.05 |
| 5 | — | — | — | — |
| 6 | 97.17 | 2.36 | 0.06 | 1.05 |

TABLE 4C

PA-6 ULTRAMID

| Sample | BHET | Dimer | Trimer | Other oligomers |
|---|---|---|---|---|
| 1 | 89.41 | 9.28 | 0.70 | 0.61 |
| 2 | 90.22 | 8.41 | 0.52 | 0.85 |
| 3 | 95.81 | 3.14 | 0.09 | 0.96 |
| 4 | 94.44 | 3.90 | 0.87 | 0.79 |
| 5 | — | — | — | — |
| 6 | 96.26 | 3.23 | 0.06 | 0.45 |

As can be seen, the process is able to guarantee a progressive improvement in the purity degree of BHET.

### Final BHET Color Analysis

To verify the overall effectiveness of the process in purifying the BHET, an analysis of the color (by UV-Vis spectrometry and according to the Cie LAB colorimetric method) was carried out on the final BHET obtained from step (1).
UV-Vis spectrophotometric analysis.

To evaluate the color characteristics of the final BHET, the total absorbance of a sample at predetermined wavelengths (Σabs₀) can be used, measured by a spectrophotometer operating in the desired wavelength range.

Usually, to cover the visible range, the selected wavelengths are: 475, 510, 570, 590, 650 nm, therefore:
Σabs₀=abs₄₇₅+abs₅₁₀+abs₅₇₀+abs₅₉₀+abs₆₅₀.

The method used for this measurement was as follows: the BHET to be analyzed is weighed and dissolved in dimethyl sulfoxide (DMSO) to obtain a BHET:DMSO weight ratio of 1:1. The solution thus obtained is placed in an optical glass test tube (optical path: 1.5 cm) and inserted into a spectrophotometer to determine the UV-Vis spectrum.

### Cie LAB Color

The color of dry BHET powder is measured using the Hunter Lab Color Scale according to the Hunter Lab L, a, b color space (details of this measurement can be found at https://support.hunterlab.com).

The measurement is performed on a cylindrical sample (diameter: 50 mm; height: 10 mm) of dry BHET powder obtained by compressing the powder in a cylindrical container at 400 bar.

The results are shown in Table 5.

TABLE 5

| | PA-6 | PA-66 | Ultramid PA-6 |
|---|---|---|---|
| | Absorbance | | |
| abs₄₇₅ | 0.009 | 0.014 | 0.009 |
| abs₅₁₀ | 0.006 | 0.008 | 0.005 |
| abs₅₇₀ | 0.003 | 0.005 | 0.004 |
| abs₅₉₀ | 0.002 | 0.004 | 0.003 |
| abs₆₅₀ | 0.001 | 0.005 | 0.002 |
| Σabs₀ | 0.021 | 0.036 | 0.023 |
| | Cie LAB Color | | |
| L | 98.08 | 98.10 | 98.62 |
| a | −0.13 | −0.11 | −0.19 |
| b | 1.51 | 1.49 | 1.10 |

The obtained values are well within the specification limits (Σabs₀<0.15; L>95; a=−0.2/+0.3; b<3).

### Example 2

A series of tests were carried out on PET waste obtained from bottles (r-PET) added with an ethylene/vinyl alcohol copolymer (EVOH) used as a barrier-polymer for food packaging (commercial product EVOH FIOIB 32% B-Pack™ EVAL—32% moles of ethylene). The added amount of EVOH was equal to 5% by weight with respect to the r-PET weight.

The PET waste added with EVOH was subjected to the glycolysis reaction under the same conditions as in Example 1. The composition of the reaction mixture was as follows:

| | weight (g) | % weight |
|---|---|---|
| r-PET | 285 | 19 |
| EG | 1200 | 80 |
| ZnAc | 0.3 | 0 |
| EVOH | 15 | 1 |
| Total | 1550.3 | 100 |

The crude BHET solution obtained from the glycolysis process was subjected to a purification process according to the present invention, carried out with methods identical to those reported for Example 1 (steps (a)-(1)).

To verify the effectiveness of the process in EVOH removal, it was not possible to use the qualitative method through the detection of the IR absorption peaks characteristic of EVOH, as the characteristic peak of EVOH is located at about 3000 nm and overlaps with the absorption peaks of BHET (located in the range 2800-3300 nm).

The analyses already described above for Example 1 were then carried out to determine the quantitative EVOH removal and the composition in terms of BHET and its oligomers in the various stages of the process, and the color analysis on the final BHET obtained from step (1), according to the methods described above. The results are reported in the following Tables 6-8:

TABLE 6

| | Separated | Separated EVOH (% by |
|---|---|---|
| Sample | EVOH (g) | weight of total EVOH) |
| A | 0.15 | 1 |
| B | 1.8 | 12 |
| C | 5.85 | 39 |
| D | 7.2 | 48 |
| E | 0 | 0 |

TABLE 7

PA-6

| Sample | BHET | Dimer | Trimer | Other oligomers |
|---|---|---|---|---|
| 1 | 89.97 | 8.83 | 0.65 | 0.55 |
| 2 | 90.32 | 8.55 | 0.56 | 0.57 |
| 4 | 96.26 | 2.77 | 0.03 | 0.94 |
| 6 | 96.64 | 2.54 | 0.06 | 0.76 |

TABLE 8

| | Absorbance |
|---|---|
| abs₄₇₅ | 0.006 |
| abs₅₁₀ | 0.006 |
| abs₅₇₀ | 0.003 |
| abs₅₉₀ | 0.003 |
| abs₆₅₀ | 0 |
| Σabs₀ | 0.018 |
| | Cie LAB Color |
| L | 98.46 |
| a | −0.14 |
| b | 0.84 |

As can be seen, also in this case the process according to the invention has proved to be effective and has made it possible to eliminate the EVOH in a quantitative way. Analogously to the polyamide Ultramid PA-6, during step (c) only a partial removal was obtained which was completed with the subsequent phases, in particular after the first treatment with PAC (phase (i)).

Furthermore, the process is able to guarantee a progressive improvement in the degree of purity and color of the BHET.

1: A process for purifying bis(2-hydroxyethyl) terephthalate (BHET), which comprises:
providing a crude BHET solution obtained from depolymerizing a polyethylene terephthalate (PET) waste by glycolysis with ethylene glycol (EG), said crude BHET solution being at a temperature from 160° C. to 220° C., and containing at least one dissolved barrier-polymer;
adding a first portion of water to said crude BHET solution in an amount such as to obtain a weight ratio EG:H₂O from 2.0 to 4.0 and cooling the solution thus obtained to a temperature from 60° C. to 90° C., so as to obtain a precipitate of said at least one barrier-polymer;
separating said precipitate of at least one barrier-polymer from the BHET solution;
adding a second portion of water to the BHET solution obtained from the separation step in an amount such as to obtain a weight ratio EG:H₂O from 0.5 to 2.5 and cooling the solution thus obtained to a temperature from 5° C. to 25° C. so as to obtain the crystallization of the BHET; and
separating the crystallized BHET from the water/EG solvent.

2: The process according to claim 1, wherein said PET waste is a multilayer product comprising at least one PET film coupled with at least one barrier-polymer film.

3: The process according to claim 1, wherein said at least one barrier-polymer is selected from the group consisting of:
(a) polyamides (PA), and copolymers thereof;
(b) polyvinyl alcohol (PVA), polyvinyl acetate (PVAc), ethylene/vinyl alcohol copolymers (EVOH), ethylene/vinyl acetate copolymers (EVA), and ethylene/vinyl acetate/vinyl alcohol terpolymers;
and mixtures thereof.

4: The process according to claim 1, wherein, after the step of separating said precipitate of at least one barrier-polymer from the BHET solution, the BHET solution thus obtained is further cooled to a temperature from 30° C. to 60° C., so as to precipitate the oligomers and optionally a further portion of said barrier-polymer.

5: The process according to claim 1, which further comprises:
after the step of separating the crystallized BHET from the water/EG solvent, dissolving the crystallized BHET in water;
treating the solution thus obtained with at least a first adsorbing agent; and
separating the at least one first adsorbing agent from the solution thus treated so as to obtain a first purified solution of BHET.

6: The process according to claim 5, which further comprises:
treating the solution obtained after the separation of the first adsorbing agent with at least one oxidizing agent at a temperature from 30° C. to 100° to obtain an oxidized solution;
treating the oxidized solution with at least a second adsorbing agent to obtain a purified oxidized solution; and
separating said at least one second adsorbing agent from the purified oxidized solution to obtain a second purified BHET solution.

7: The process according to claim 6, wherein
said at least one oxidizing agent is an inorganic oxidizing agent selected from the group consisting of: alkali metal or alkaline earth metal chlorites or hypochlorites; alkali metal or alkaline earth metal persalts; potassium permanganate; hydrogen peroxide; ozone; gaseous chlorine; and mixtures thereof.

8: The process according to claim 6, wherein said at least one first adsorbing agent and said at least one second adsorbing agent, identical or different from each other, are an activated carbon or a silica.

9: The process according to claim 1, wherein said crude BHET solution is provided at a temperature from 190° C. to 210° C.

10: The process according to claim 1, wherein said first portion of water is added to said crude BHET solution in an amount such as to obtain a weight ratio EG:H₂O from 2.5 to 3.5.

11: The process according to claim 1, wherein the solution obtained by adding said first portion of water to said crude BHET solution is cooled to a temperature from 65° C. to 80° C.

12: The process according to claim 1, wherein said second portion of water is added to the BHET solution obtained from the separation step in an amount such as to obtain a weight ratio EG:H₂O from 1.0 to 2.0.

13: The process according to claim 1, wherein the solution obtained by adding said second portion of water to the BHET solution obtained from the separation step is cooled to a temperature from 10° C. to 20° C.
